(19) United States
(12) Patent Application Publication
(10) Pub. No.: US 2019/0175301 A1
(43) Pub. Date: Jun. 13, 2019

(54) NEAR-NFRARED IMAGING APPARATUS AND MARKER MEMBER FOR NEAR-INFRARED IMAGING APPARATUS

(71) Applicant: SHIMADZU CORPORATION, Kyoto-shi, Kyoto (JP)

(72) Inventor: Akihiro ISHIKAWA, Kyoto-shi (JP)

(73) Assignee: Shimadzu Corporation, Kyoto-shi, Kyoto (JP)

(21) Appl. No.: 16/302,770

(22) PCT Filed: Jun. 9, 2016
(86) PCT No.: PCT/JP2016/067275

§ 371 Date: Nov. 19, 2018

(51) Int. Cl. A61B 90/00; A61B 5/00

(52) U.S. Cl. CPC A61B 90/39; A61B 2090/395; A61B 5/0071

(57) ABSTRACT

A near-infrared imaging apparatus according to the present invention comprises: an excitation light source unit that irradiates a fluorescent agent within a patient P with near-infrared excitation light; a marker member including a marker light source unit that generates near-infrared light; a near-infrared sensor that detects near-infrared light and near-infrared fluorescent light generated by the fluorescent agent; and an image forming unit that captures an image of the detected near-infrared light and near-infrared fluorescent light.

### TECHNICAL FIELD

This invention relates to a near infrared imaging apparatus, and particularly relates to a near infrared imaging apparatus including a near infrared detector that detects near infrared light generated from a fluorescent agent in a test object, and a marker member for the near infrared imaging apparatus.

### BACKGROUND ART

Conventionally, a near infrared imaging apparatus including a near infrared detector that detects near infrared light generated from a fluorescent agent in a test object has been known. For example, such a near infrared imaging apparatus is disclosed in JP-A-2015-188559.

A medical imaging apparatus disclosed in the above-mentioned JP-A-2015-188559 includes an illumination unit, an imaging unit, and a surgeon observation monitor. Further, the medical imaging apparatus (near infrared imaging apparatus) is configured as apart of an intraoperative support apparatus. Further, this medical imaging apparatus is configured such that a contrast medium injected into a blood vessel of a patient is irradiated with excitation light of a near infrared ray from the illumination unit during surgery. Further, the imaging unit is configured to capture an image of fluorescence in an infrared region generated by irradiating the contrast medium with excitation light. Further, the surgeon observation monitor is configured such that the image captured by the imaging unit is displayed.

In addition, when the conventional medical imaging apparatus disclosed in the above-mentioned JP-A-2015-188559 is used, a surgeon places a mark at (marks) a position on a skin of the patient corresponding to a path of a lymph duct using a pen-shaped marker member while checking an image of a blood vessel or the lymph duct displayed on the surgeon observation monitor after injecting a contrast medium into the blood vessel or the lymph duct of the patient and before determining an incision site of the patient, in some cases.

### CITATION LIST

### Patent Document

Patent Document 1: JP-A-2015-188559

### SUMMARY OF THE INVENTION

### Technical Problem

However, in the conventional medical imaging apparatus (near infrared imaging apparatus) disclosed in the above-mentioned JP-A-2015-188559, when the surgeon places a mark on the patient, the surgeon needs to place the mark on an actual skin of the patient by relying on the image of the blood vessel or the lymph duct while visually recognizing the image of the blood vessel or the lymph duct displayed on the surgeon observation monitor. However, the conventional marker member has a problem that the marker member is not displayed on the surgeon observation monitor and a position at which the mark is actually placed may be shifted from a position at which the mark needs to be placed.

The invention has been made to solve the above-mentioned problem, and an object of the invention is to provide a near infrared imaging apparatus capable of inhibiting a position at which a mark is actually placed from being shifted from a position at which the mark needs to be placed when the mark is placed on a test object using a marker member, and a marker member for the near infrared imaging apparatus.

### Solution to Problem

To achieve the object, a near infrared imaging apparatus according to a first aspect of the invention includes a first light source unit that irradiates a test object-side fluorescent agent inside a test object with near infrared excitation light, a marker member that includes a near infrared ray generator for generating first near infrared light and is used to place a mark on the test object, a near infrared detector that detects the first near infrared light and detects second near infrared light generated from the test object-side fluorescent agent by the irradiated near infrared excitation light, and an imaging unit that images the first near infrared light and the second near infrared light detected by the near infrared detector. In the specification, a “near infrared ray” means light having a longer wavelength than that of a visible ray, and is described to mean, for example, light having a wavelength within a range of 700 nm or more and 900 nm or less.

In the near infrared imaging apparatus according to the first aspect of the invention, as described above, the near infrared ray generator that generates the first near infrared light is included, and the marker member for placing the mark on the test object is provided. Further, the near infrared detector is configured to detect the first near infrared light and detect the second near infrared light generated from the test object-side fluorescent agent by the irradiated near infrared excitation light. In addition, the imaging unit is configured to image the first near infrared light and the second near infrared light detected by the near infrared detector. In this way, it is possible to allow a user to visually recognize an image in a state in which an image corresponding to a position of the marker member held by the user (surgeon) is superimposed on an image of the test object-side fluorescent agent (blood vessel or lymph duct of the patient). As a result, it is possible to allow the user to visually recognize an image of the test object-side fluorescent agent related to a position at which the mark needs to be placed and an image indicating a position of the marker member used to actually place the mark together. Therefore, when the mark is placed on the test object by the marker member, it is possible to inhibit the position at which the mark is actually placed from being shifted from the position at which the mark needs to be placed.

In the near infrared imaging apparatus according to the first aspect, preferably, the marker member includes a holding portion corresponding to a portion held by a user and a pen tip disposed on the test object, and the near infrared ray generator is provided around the pen tip of the marker member. According to this configuration, it is possible to image an image indicating a position around the pen tip of the marker member disposed on the test object. Thus, it is possible to effectively inhibit the position at which the mark is actually placed by the pen tip of the marker member from being shifted from the position on the test object at which the mark needs to be placed.

In the near infrared imaging apparatus according to the first aspect, preferably, the near infrared ray generator is formed to have at least one of an arrow shape, a rectangular shape, or a circular shape. According to this configuration, the first near infrared light is imaged in a state of having an arrow shape, a rectangular shape, or a circular shape, and thus it is possible to improve discrimination as an image indicating the position of the marker member.

In the near infrared imaging apparatus according to the first aspect, preferably, the near infrared ray generator includes a second light source unit that irradiates the near infrared detector with the first near infrared light. According to this configuration, it is possible to easily generate the first near infrared light by providing the second light source unit in the marker member.

In this case, preferably, a switching unit that switches between a state in which power is supplied to the second light source unit and a state in which the power is not supplied to the second light source unit is further included. According to this configuration, it is possible to switch between a state in which the second light source unit is turned ON and a state in which the second light source unit is turned OFF according to a need of the user, and thus it is possible to improve convenience during use of the marker member.

In the near infrared imaging apparatus according to the first aspect, preferably, the near infrared ray generator includes a marker-side fluorescent agent that generates the first near infrared light by being irradiated with the near infrared excitation light from the first light source unit. According to this configuration, unlike the case of providing the second light source unit, a structure for supplying power to the near infrared ray generator is unnecessary, and thus it is possible to inhibit a structure of the near infrared ray generator from being complicated.

In the near infrared imaging apparatus according to the first aspect, preferably, the near infrared ray generator includes a detachable portion for attachment and detachment to and from the marker member. According to this configuration, using the detachable portion, the near infrared ray generator may be removed from the used marker member, and the near infrared ray generator may be attached to a new marker member. That is, when the consumable marker member is replaced, the near infrared ray generator can be reused.

In the near infrared imaging apparatus according to the first aspect, preferably, a wavelength of the first near infrared light is a wavelength in a vicinity of a wavelength of the second near infrared light. According to this configuration, the near infrared detector capable of detecting the second near infrared light may detect the first near infrared light from the near infrared ray generator. In this way, even in the case of using a conventional near infrared detector capable of detecting the second near infrared light from the test object-side fluorescent agent, it is possible to detect the first near infrared light from the marker member of the invention.

In the near infrared imaging apparatus according to the first aspect, preferably, the near infrared imaging apparatus is used as a medical imaging apparatus. In addition, preferably, the near infrared imaging apparatus is used as an intraoperative support apparatus. According to this configuration, when the surgeon places a mark on the patient using the marker member during surgery, it is possible to inhibit a position at which the mark is actually placed from being shifted from a position at which the mark needs to be placed. Thus, applying the near infrared imaging apparatus of the invention to the medical imaging apparatus or the intraoperative support apparatus is particularly effective.

In the near infrared imaging apparatus according to the first aspect, preferably, the near infrared imaging apparatus further includes a third light source unit that irradiates the test object with visible light, and a visible light detector that detects the visible light irradiated by the third light source unit and reflected by the test object, and the imaging unit includes an image composition unit that composes a near infrared light image obtained by imaging the first near infrared light and the second near infrared light detected by the near infrared detector and a visible light image obtained by imaging the visible light detected by the visible light detector. According to this configuration, it is possible to allow the user to visually recognize the position at which the mark is placed (marked position) using the visible light image. As a result, it is possible to allow the user to visually recognize the visible light image indicating the position at which the mark is placed while allowing the user to visually recognize the near infrared light image indicating the position of the marker member corresponding to the position at which the mark is actually placed.

A marker member for a near infrared imaging apparatus according to a second aspect of the invention is a marker member used for the near infrared imaging apparatus including a first light source unit that irradiates a test object-side fluorescent agent inside a test object with near infrared excitation light, a near infrared detector that detects second near infrared light generated from the test object-side fluorescent agent by the irradiated near infrared excitation light, and an imaging unit that images the second near infrared light detected by the near infrared detector, wherein the marker member includes a near infrared ray generator that generates first near infrared light detectable by the near infrared detector.

In the marker member for the near infrared imaging apparatus according to the second aspect of the invention, as described above, the near infrared ray generator that generates the first near infrared light detectable by the near infrared detector is included. In this way, it is possible to provide the marker member for the near infrared imaging apparatus capable of inhibiting the position at which the mark is actually placed from being shifted from the position at which the mark needs to be placed.

### Advantageous Effects of the Invention

According to the invention, as described above, when a mark is placed on a test object using a marker member, it is possible to inhibit a position at which the mark is actually placed from being shifted from a position at which the mark needs to be placed.

### BRIEF DESCRIPTION OF THE DRAWINGS

FIG. 1 is a block diagram of a near infrared imaging apparatus according to a first embodiment of the invention.

FIG. 2 is a diagram for description of an image displayed on a display unit of the near infrared imaging apparatus according to the first embodiment of the invention.

FIG. 3 is a schematic diagram of an entire configuration of the near infrared imaging apparatus according to the first embodiment of the invention.

FIG. 4 is a side view of a marker member of the near infrared imaging apparatus according to the first embodiment of the invention.

FIG. 5 is a diagram (cross-sectional view) illustrating a state in which the marker member of the near infrared imaging apparatus according to the first embodiment of the invention generates near infrared light.

FIG. 6 is a diagram specifically illustrating the image displayed on the display unit of the near infrared imaging apparatus according to the first embodiment of the invention.

FIG. 7 is a diagram (cross-sectional view) illustrating a state in which a marker member of a near infrared imaging apparatus according to a second embodiment of the invention generates near infrared fluorescence.

FIG. 8 is a partial side view illustrating a marker member of a near infrared imaging apparatus according to a third embodiment of the invention.

FIG. 9 is a diagram for description of attachment and detachment of a detachable portion to and from the marker member of the near infrared imaging apparatus according to the third embodiment of the invention.

FIG. 10 is a block diagram of a near infrared imaging apparatus according to a first modified example of the first to third embodiments of the invention.

FIG. 11 is a diagram (cross-sectional view) illustrating a configuration of a marker member of a near infrared imaging apparatus according to a second modified example of the first embodiment and the third embodiment of the invention.

FIG. 12 is a schematic diagram illustrating a configuration of a near infrared imaging apparatus according to a third modified example of the first to third embodiments of the invention.

### MODE FOR CARRYING OUT THE INVENTION

Hereinafter, embodiments embodying the invention will be described based on drawings.

### First Embodiment

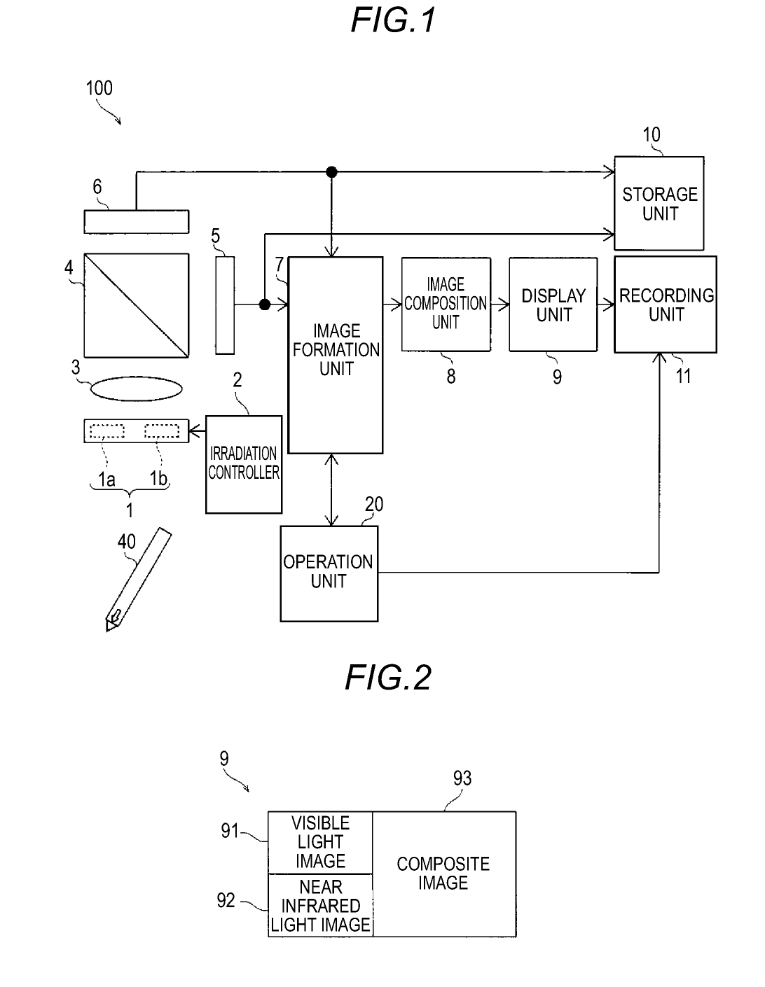

A near infrared imaging apparatus 100 according to a first embodiment of the invention will be described with reference to FIG. 1 to FIG. 6. In addition, in the first embodiment, for example, the near infrared imaging apparatus 100 is used (configured) as a medical imaging apparatus used for angiography and lymphangiography during surgical operation. In addition, the near infrared imaging apparatus 100 is used (configured) as apart of an intraoperative support apparatus (intraoperative support system) used during surgery. In the specification of the present application, a “near infrared ray” means light having a longer wavelength than that of a visible ray, and is described to mean, for example, light having a wavelength within a range of 700 nm or more and 900 nm or less.

Specifically, the near infrared imaging apparatus 100 is used to allow a user such as a surgeon Q to check positions or paths of a blood vessel or a lymph duct and a lymph node of a contrasted patient P (test object) in a surgery of a breast cancer sentinel lymph node.

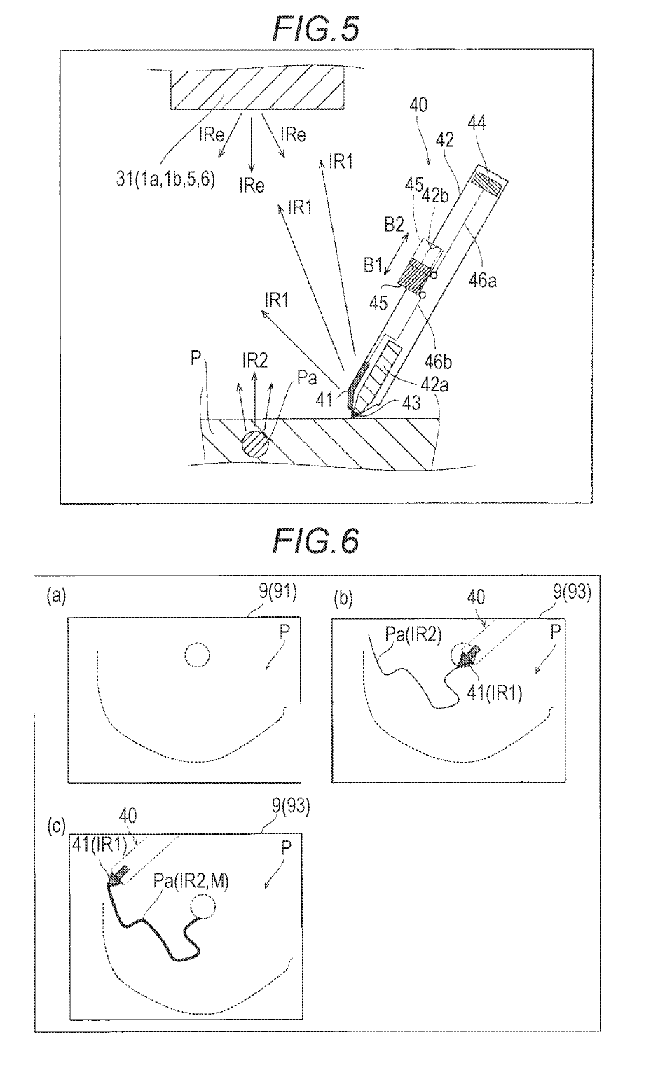

As illustrated in FIG. 1, the near infrared imaging apparatus 100 includes a light source unit 1. The light source unit 1 includes, for example, a light emitting diode (LED). The light source unit 1 includes a white light source unit 1a that irradiates the patient P with white light including visible light and an excitation light source unit 1b that irradiates a fluorescent agent Pa (see FIG. 5) (contrast medium) inside a body of the patient P with near infrared excitation light (hereinafter “excitation light IRe”). The white light source unit 1a is an example of a “third light source unit” in claims. In addition, the excitation light source unit 1b is an example of a “first light source unit” in the claims. In addition, the fluorescent agent Pa is an example of a “test object-side fluorescent agent” in the claims.

Specifically, for example, the fluorescent agent Pa is made of indocyanine green (ICG) which is a fluorescent pigment. Further, the excitation light IRe is near infrared light having a wavelength of 750 nm or more and 820 nm or less. For example, the excitation light IRe is near infrared light having a wavelength of about 810 nm. When indocyanine green is irradiated with the excitation light IRe, near infrared fluorescence IR2 having a wavelength of about 845 nm is generated from indocyanine green. In addition, white light irradiated from the white light source unit 1a is reflected from a skin surface of the patient P as reflected light (visible light). The near infrared fluorescence IR2 is an example of “second near infrared light” in the claims.

In addition, the near infrared imaging apparatus 100 includes an irradiation controller 2. The irradiation controller 2 is configured as a control circuit and configured to control irradiation of light (white light, excitation light IRe) from the light source unit 1, stop of irradiation, etc. based on an input operation by an operation unit 20 described below.

In addition, a zoom lens 3 is provided around the light source unit 1. Reflected light (visible light) from the skin surface of the patient P, near infrared fluorescence IR2 generated from the fluorescent agent Pa, and near infrared light IR1 from a marker light source unit 41 described below enter the zoom lens 3. The near infrared light IR1 is an example of “first near infrared light” in the claims.

In addition, a prism 4 is provided around the zoom lens 3. Light from the zoom lens 3 enters the prism 4. Further, the prism 4 has a function of separating reflected light (visible light) from the skin surface of the patient P, the near infrared light IR1, and the near infrared fluorescence IR2.

In addition, the near infrared imaging apparatus 100 includes a visible light sensor 5 that detects visible light separated by the prism 4. For example, the visible light sensor 5 includes a charge coupled device (CCD). Further, the visible light sensor 5 is an example of a “visible light detector” in the claims.

In addition, the near infrared imaging apparatus 100 includes a near infrared sensor 6 that detects the near infrared fluorescence IR2 generated by the excitation light IRe irradiated from the excitation light source unit 1b and the near infrared light IR1 from the marker light source unit 41. For example, the near infrared sensor 6 is configured to be able to detect a near infrared ray having a wavelength within a range of 780 nm or more and 860 nm or less. For example, the near infrared sensor 6 includes a CCD or a photomultiplier tube. The near infrared sensor 6 is an example of a “near infrared detector” in the claims.

In addition, the near infrared imaging apparatus 100 includes an image formation unit 7. The visible light detected by the visible light sensor 5 and the near infrared light IR1 and the near infrared fluorescence IR2 detected by the near infrared sensor 6 are input to the image formation unit 7. Then, the image formation unit 7 forms the visible light detected by the visible light sensor 5 as a 24-bit (=3×8) image including three colors of RGB (red, green, and blue). In addition, the image formation unit 7 forms the near infrared light IR1 and the near infrared fluorescence IR2 detected by the near infrared sensor 6 as an 8-bit image. The image formation unit 7 is an example of an “imaging unit” in the claims.

In addition, the near infrared imaging apparatus 100 includes an image composition unit 8. Here, in the first embodiment, as illustrated in FIG. 2, the image composition unit 8 is configured to form a composite image 93 by composing a visible light image 91 obtained by imaging the visible light and a near infrared light image 92 obtained by imaging the near infrared light IR1 and the near infrared fluorescence IR2. For example, each of the image formation unit 7 and the image composition unit 8 is configured as an image processing circuit. In addition, the image composition unit 8 is an example of the “imaging unit” in the claims.

In addition, the near infrared imaging apparatus 100 includes a display unit 9. As illustrated in FIG. 2, the visible light image 91, the near infrared light image 92, and the composite image 93 are displayed on the display unit 9.

In addition, as illustrated in FIG. 1, the near infrared imaging apparatus 100 includes a storage unit 10. The storage unit 10 includes a storage element, etc. and is configured to save the visible light (signal) detected by the visible light sensor 5 and the near infrared light IR1 and the near infrared fluorescence IR2 (signal) detected by the near infrared sensor 6.

In addition, a recording unit 11 is provided in the near infrared imaging apparatus 100. The recording unit 11 includes a storage element, etc. and is configured to record an image displayed on the display unit 9.

In addition, the near infrared imaging apparatus 100 includes an operation unit 20. The operation unit 20 is configured to receive an input operation to the near infrared imaging apparatus 100 by the user (surgeon Q, etc.). In addition, the operation unit 20 includes a control circuit. Further, the operation unit 20 is configured to operate (control) irradiation of light from the light source unit 1, stop of irradiation, adjustment of brightness and sensitivity, a method of displaying an image displayed on the display unit 9, etc. based on the input operation.

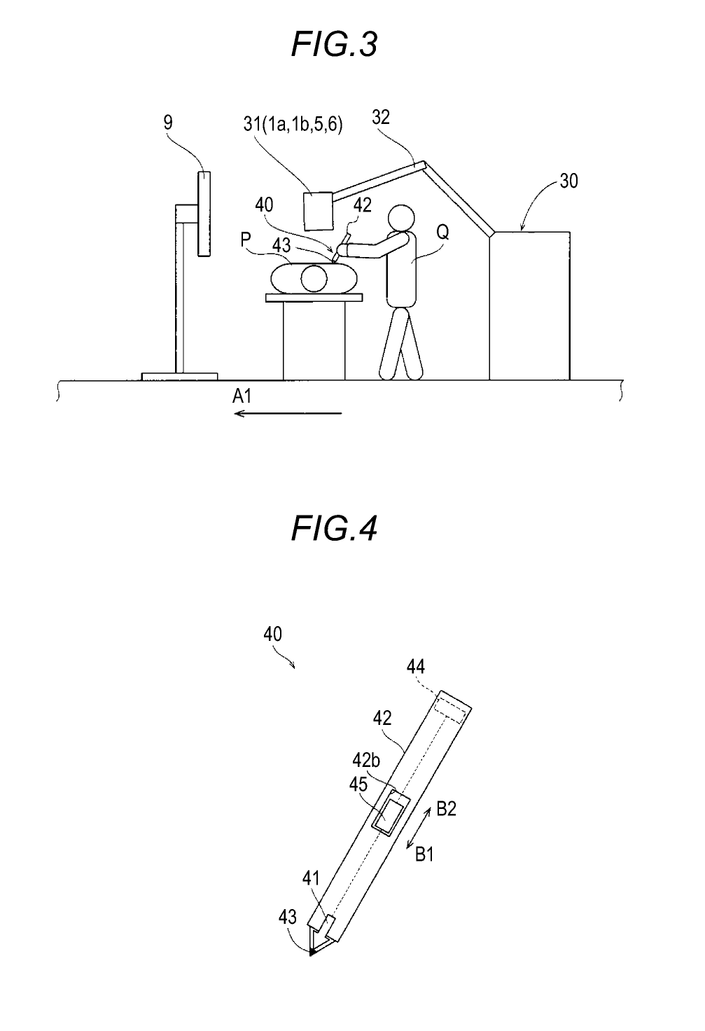

In addition, as illustrated in FIG. 3, the near infrared imaging apparatus 100 includes a main apparatus body 30 provided with the white light source unit 1a, the excitation light source unit 1b, the visible light sensor 5, the near infrared sensor 6, etc. The white light source unit 1a, the excitation light source unit 1b, the visible light sensor 5, the near infrared sensor 6, etc. are disposed inside an illumination/photographing unit 31. In addition, an arm 32 is provided on the main apparatus body 30. The illumination/photographing unit 31 is attached to the arm 32, and the illumination/photographing unit 31 is configured to be movable.

In addition, the display unit 9 is provided separately from the main apparatus body 30. For example, the display unit 9 is disposed in a facing direction of the surgeon Q (user) (on an arrow A1 direction side), and is disposed at a height position at which an image displayed on the display unit 9 is visually recognizable when the surgeon Q performs treatment on the patient P (test object).

Here, in the first embodiment, the near infrared imaging apparatus 100 is provided separately from the main apparatus body 30 and used around the patient P, and includes a marker member 40 for placing a mark on the skin of the patient P (test object). Further, the marker member 40 includes the marker light source unit 41 that irradiates the near infrared sensor 6 with the near infrared light IR1. The marker light source unit 41 is an example of a “near infrared ray generator” and a “second light source unit” in the claims.

Specifically, as illustrated in FIG. 3, the marker member 40 is configured as a surgical marker (skin marker). Further, the marker member 40 includes a main marker body 42 corresponding to a portion held by the surgeon Q and a pen tip 43 disposed on the skin of the patient P (disposed on the patient P side in relation to the main marker body 42) during use. The main marker body 42 is an example of a “holding portion” in the claims.

As illustrated in FIG. 4, for example, the main marker body 42 is formed to have a cylindrical shape. In addition, as illustrated in FIG. 5, an ink tank 42a is provided inside the main marker body 42. The ink tank 42a is configured to supply marker ink to the pen tip 43.

The pen tip 43 is configured such that the marker ink is applied to the skin of the patient P by being brought into contact with the skin of the patient P. Further, when the marker ink is applied to the skin of the patient Q using the marker member 40, a treatment of placing a mark (marking) is performed.

Here, in the first embodiment, as illustrated in FIG. 4, the marker light source unit 41 is provided around the pen tip 43 of the marker member 40. Further, the marker light source unit 41 is formed to have an arrow shape. For example, the marker light source unit 41 is configured to have an arrow shape in which an arrow tip end is directed to the pen tip 43 side in a side view (see FIG. 4) and such that an entire arrow-shaped portion emits light.

Further, for example, the marker light source unit 41 includes an LED. In this way, the marker light source unit 41 is configured to be able to emit the near infrared light IR1 by being supplied with power. Further, the marker light source unit 41 is configured to generate the near infrared light IR1 detectable by the near infrared sensor 6. For example, the marker light source unit 41 is configured to generate near infrared light IR1 having a wavelength of about 845 nm. That is, the wavelength of the near infrared light IR1 emitted by the marker light source unit 41 is a wavelength in the vicinity of (substantially the same as) the wavelength of the near infrared fluorescence IR2 emitted by the fluorescent agent Pa.

Further, as illustrated in FIG. 5, the near infrared sensor 6 provided inside the illumination/photographing unit 31 is configured to detect the near infrared light IR1 from the marker light source unit 41 and the near infrared fluorescence IR2 from the fluorescent agent Pa inside the patient P.

In addition, a battery 44, a switch 45, and wires 46a and 46b are provided in the marker member 40. The battery 44 is provided inside the main marker body 42 and disposed on an opposite side from the pen tip 43 of the main marker body 42. In addition, the wire 46a is connected to the battery 44. The wire 46 is connected to the marker light source unit 41. Further, the battery 44 is configured to supply power to the marker light source unit 41 through the wires 46a and 46b.

Here, in the first embodiment, the switch 45 is configured to switch between a state in which power from the battery 44 is supplied to the marker light source unit 41 and a state in which power from the battery 44 is not supplied to the marker light source unit 41.

For example, as illustrated in FIG. 4, an opening 42b is provided in the main marker body 42, and the switch 45 is disposed to protrude to an outside of the main marker body 42 from the opening 42b. Further, the switch 45 is configured to be slidable in a direction in which the main marker body 42 extends (arrow B1 direction and arrow B2 direction). Further, as illustrated in FIG. 5, the switch 45 contains a conductor and is configured to connect the wire 46a and the wire 46b to each other in a state of being disposed on the arrow B1 direction side. In addition, the switch 45 is configured to electrically disconnect the wire 46a and the wire 46b from each other in a state of being disposed on the arrow B2 direction side.

That is, the marker member 40 is configured to be able to switch ON and OFF the marker light source unit 41 by a switching operation of the switch 45 by the surgeon Q.

Next, a specific use example of the marker member 40 according to the first embodiment will be described with reference to FIG. 6. A description will be given of an example in which a position of a blood vessel or a lymph duct of the patient P (test object) is marked by the surgeon Q using the marker member 40 in a surgery of abreast cancer sentinel lymph node. For example, an example is shown in which a position on the skin surface corresponding to the position and the path of the lymph duct into which the fluorescent agent Pa is injected is marked as a position at which a mark needs to be placed.

FIG. 6(a) illustrates a state in which the visible light image 91 obtained by imaging reflected light reflected from the patient P is displayed on the display unit 9. Further, FIG. 6(b) illustrates a state in which the composite image 93 of a state in which the fluorescent agent Pa is injected into the lymph duct (lymph node) of the patient P is displayed on the display unit 9. That is, in FIG. 6(b), the near infrared fluorescence IR2 from the fluorescent agent and the near infrared light IR1 from the marker light source unit 41 of the marker member 40 are imaged.

Further, the surgeon Q (not illustrated in FIG. 6) performs a treatment of placing a mark on the skin of the patient P corresponding to the imaged lymph duct while visually recognizing an image (display of an arrow shape) of the near infrared light IR1 and an image of the near infrared fluorescence IR2 displayed on the display unit 9 in a state of holding the marker member 40. In this way, as illustrated in FIG. 6(c), the mark (thick line portion of reference symbol M of FIG. 6) is placed on the skin of the patient P at a position corresponding to the lymph duct. Then, as illustrated in FIG. 6(c), the position (reference symbol M) at which the mark is actually placed is inhibited from being shifted from a position at which the mark needs to be placed (position of the fluorescent agent Pa in the first embodiment).

### Effects of First Embodiment

In the first embodiment, it is possible to obtain the following effects.

In the first embodiment, as described above, in the near infrared imaging apparatus 100, the marker light source unit 41 for generating the near infrared light IR1 is included, and the marker member 40 for placing a mark on the skin of the patient P is provided. Further, the near infrared sensor 5 is configured to detect the near infrared light IR1 and detect the near infrared fluorescence IR2 generated from the fluorescent agent Pa by the irradiated excitation light IRe. In addition, the image formation unit 7 is configured to image the near infrared light IR1 and the near infrared fluorescence IR2 detected by the near infrared sensor 6. In this way, it is possible to allow the user (surgeon Q) to visually recognize an image in a state in which an image corresponding to a position of the marker member 40 held by the user is superimposed on an image of the fluorescent agent Pa (blood vessel or lymph duct of the patient P). As a result, it is possible to allow the user to visually recognize an image of the fluorescent agent Pa related to the position at which the mark needs to be placed and an image indicating the position of the marker member 40 used to actually place the mark together (see FIG. 6). Therefore, when the mark is placed on the skin of the patient P using the marker member 40, it is possible to inhibit the position at which the mark is actually placed from being shifted from the position at which the mark needs to be placed.

In addition, in the first embodiment, as described above, the main marker body 42 corresponding to a portion held by the user and the pen tip 43 disposed on the test object (on the skin of the patient P) are provided in the marker member 40. In addition, the marker light source unit 41 is provided around the pen tip 43 of the marker member 40. In this way, it is possible to image an image indicating a position around the pen tip 43 of the marker member 40 relatively close to the test object. Thus, it is possible to effectively inhibit the position at which the mark is actually placed by the pen tip 43 of the marker member 40 from being shifted from the position on the test object at which the mark needs to be placed.

In addition, in the first embodiment, as described above, the marker light source unit 41 is formed to have an arrow shape. In this way, the near infrared light IR1 is imaged in a state having an arrow shape (see FIG. 6), and thus it is possible to improve discrimination as an image indicating the position of the marker member 40.

In addition, in the first embodiment, as described above, the marker light source unit 41 is configured to irradiate the near infrared sensor 6 with the near infrared light IR1. In this way, it is possible to easily generate the near infrared light IR1 by providing the marker light source unit 41 in the marker member 40.

In addition, in the first embodiment, as described above, the marker member 40 is provided with the switch 45 that switches between the state in which power is supplied to the marker light source unit 41 and the state in which power is not supplied to the marker light source unit 41. In this way, it is possible to switch between a state in which the marker light source unit 41 is turned ON and a state in which the marker light source unit 41 is turned OFF according to a need of the user, and thus it is possible to improve convenience during use of the marker member 40.

In addition, in the first embodiment, as described above, the wavelength of the near infrared light IR1 is configured to be a wavelength in the vicinity of the wavelength of the near infrared fluorescence IR2. In this way, the near infrared light IR1 from the marker light source unit 41 may be detected by the near infrared sensor 6 capable of detecting the near infrared fluorescence IR2. In this way, even in the case of using a conventional near infrared sensor capable of detecting the near infrared fluorescence IR2 from the fluorescent agent Pa, it is possible to detect the near infrared light IR1 from the marker member 40 of the first embodiment.

In addition, in the first embodiment, as described above, the near infrared imaging apparatus 100 is used as the medical imaging apparatus. In addition, the near infrared imaging apparatus 100 is used as the intraoperative support apparatus. In this way, when the surgeon Q places a mark on the patient P using the marker member 40 during surgery, it is possible to inhibit a position at which the mark is actually placed from being shifted from a position at which the mark needs to be placed. Thus, the first embodiment in which the near infrared imaging apparatus 100 is applied to the medical imaging apparatus or the intraoperative support apparatus is particularly effective.

In addition, in the first embodiment, as described above, the light source unit 1 is provided with the white light source unit 1a that irradiates the test object with white light having visible light. In addition, the near infrared imaging apparatus 100 is provided with the visible light sensor 5 that detects visible light irradiated by the white light source unit la and reflected by the test object. In addition, the near infrared imaging apparatus 100 is provided with the image composition unit 8 that generates the composite image 93 by composing the near infrared light image 92 obtained by imaging the near infrared light IR1 and the near infrared fluorescence IR2 detected by the near infrared sensor 6 and the visible light image 91 obtained by imaging visible light detected by the visible light sensor 5. In this way, it is possible to allow the user to visually recognize the position at which the mark is placed (reference symbol M of FIG. 6) using the visible light image 91. As a result, it is possible to allow the user to visually recognize the visible light image 91 indicating the position at which the mark is placed while allowing the user to visually recognize the near infrared light image 92 indicating the position of the marker member 40 corresponding to the position at which the mark is actually placed.

### Second Embodiment

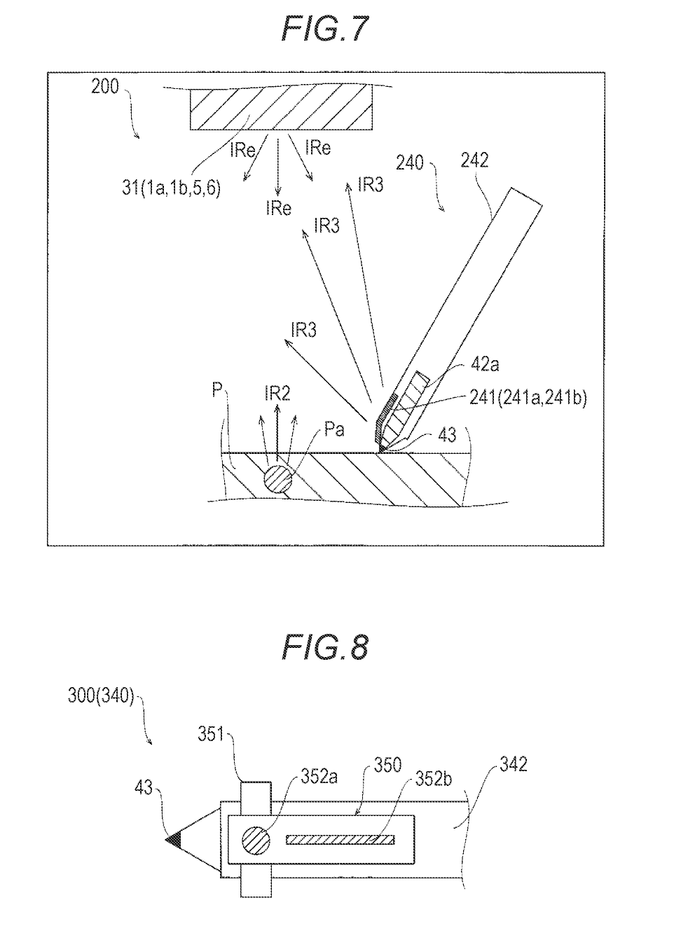

Next, a description will be given of a configuration of a near infrared imaging apparatus 200 according to a second embodiment with reference to FIG. 7. In a marker member 240 of the near infrared imaging apparatus 200 according to the second embodiment, unlike the marker member 40 configured to be able to irradiate the near infrared light IR1 using the marker light source unit 41, near infrared fluorescence IR3 may be generated by a marker-side fluorescent agent 241a of a marker-side fluorescent portion 241. The same reference symbol will be assigned to the same component as that of the first embodiment, and a description thereof will be omitted.

As illustrated in FIG. 7, the near infrared imaging apparatus 200 according to the second embodiment includes the marker member 240. Further, the marker member 240 includes the marker-side fluorescent portion 241. Further, the marker-side fluorescent portion 241 includes the marker-side fluorescent agent 241a that generates the near infrared fluorescence IR3 by being irradiated with excitation light IRe from an excitation light source unit 1b of a light source unit 1. The marker-side fluorescent portion 241 is an example of the “near infrared ray generator” in the claims. In addition, the near infrared fluorescence IR3 is an example of the “first near infrared light”.

Specifically, the marker-side fluorescent agent 241a is made of indocyanine green. That is, the marker-side fluorescent agent 241a is the same fluorescent agent as a fluorescent agent Pa injected into the patient P. In this way, the marker-side fluorescent portion 241 is configured to be able to generate the near infrared fluorescence IR3 having a wavelength of about 845 nm which can be detected by a near infrared sensor 6 by the marker-side fluorescent agent 241a being irradiated with the excitation light IRe from the excitation light source unit 1b.

Specifically, as illustrated in FIG. 7, the marker-side fluorescent portion 241 includes a member 241b containing (coated with) the marker-side fluorescent agent 241a. The marker-side fluorescent portion 241 is attached to a pen tip 43 side of a main marker body 242.

Further, the near infrared imaging apparatus 200 is configured to image the near infrared fluorescence IR3 indicating a position of the marker member 240 and image near infrared fluorescence IR2 from the fluorescent agent Pa inside the patient P.

In addition, other components of the near infrared imaging apparatus 200 according to the second embodiment are the same as those of the near infrared imaging apparatus 100 according to the first embodiment.

### Effects of Second Embodiment

In the second embodiment, it is possible to obtain the following effects.

In addition, in the second embodiment, as described above, the marker-side fluorescent portion 241 includes the marker-side fluorescent agent 241a that generates the near infrared fluorescence IR3 by being irradiated with the excitation light IRe from the first light source unit. In this way, unlike the case of providing the marker light source unit 41 as in the first embodiment, a structure (the battery 44, etc.) for supplying power to the marker light source unit 41 is unnecessary, and thus it is possible to inhibit a structure of the marker member 40 from being complicated.

In addition, other effects of the near infrared imaging apparatus 200 according to the second embodiment are the same as those of the near infrared imaging apparatus 100 in the first embodiment.

### Third Embodiment

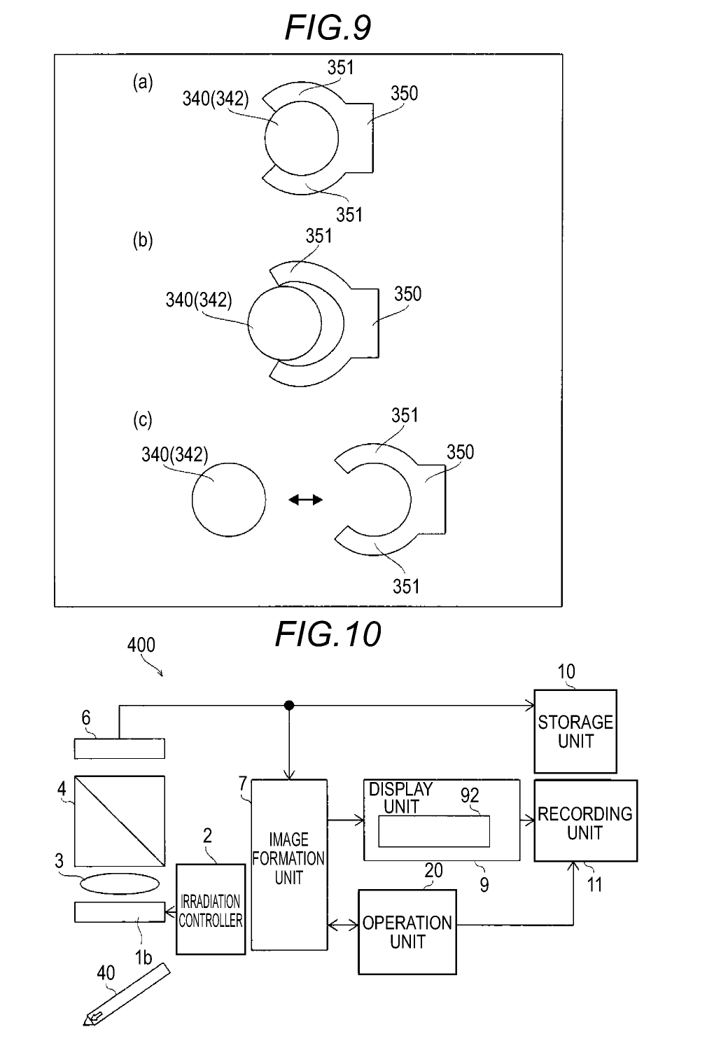

Next, a description will be given of a configuration of a near infrared imaging apparatus 300 according to a third embodiment with reference to FIG. 8 and FIG. 9. In the near infrared imaging apparatus 300 according to the third embodiment, a near infrared light emitting member 350 is provided with a detachable portion 351 for attachment and detachment to and from a marker member 340. The same reference symbol will be assigned to the same component as that of the first embodiment and the second embodiment, and a description thereof will be omitted.

As illustrated in FIG. 8, the near infrared imaging apparatus 300 according to the third embodiment includes the marker member 340. Further, the near infrared ray generating member 350 including a marker-side light source unit 352a having a circular shape (dot shape) capable of irradiating near infrared light IR1 and a marker-side light source unit 352b having a rectangular shape (linear shape) is attached to the marker member 340.

Here, in the third embodiment, as illustrated in FIG. 9, the near infrared ray generating member 350 includes the detachable portion 351 for attachment and detachment to and from the marker member 340. The detachable portion 351 is configured to have a shape of a pair of hooks. As illustrated in FIG. 9(a), the detachable portion 351 is formed to cover a part of an outer circumference of a main marker body 342 in a state of being attached to the marker member 340. Further, the detachable portion 351 is fixed to the marker member 340 by pressing of a part thereof in the shape of the pair of hooks in a direction approaching each other.

In addition, as illustrated in FIG. 9(b), the part of the detachable portion 351 in the shape of the pair of hooks is made of an elastically deformable material, and the near infrared ray generating member 350 can be detached from the marker member 340 as illustrated in FIG. 9(c) by the part in the shape of the pair of hooks being pulled apart to move away from each other. In addition, in the third embodiment, from a state in which the near infrared ray generating member 350 is detached from the marker member 340 as illustrated in FIG. 9(c), the part of the detachable portion 351 in the shape of the pair of hooks is elastically deformed as illustrated in FIG. 9(b), thereby allowing the near infrared ray generating member 350 to be attached to the marker member 340 again as illustrated in FIG. 9(a).

In addition, other components of the near infrared imaging apparatus 300 according to the third embodiment are the same as those of the near infrared imaging apparatus 100 according to the first embodiment.

### Effects of Third Embodiment

In the third embodiment, it is possible to obtain the following effects.

In addition, in the third embodiment, as described above, the detachable portion 351 for attachment and detachment to and from the marker member 340 is provided in the near infrared ray generating member 350. In this way, using the detachable portion 351, the near infrared ray generating member 350 may be removed from the used marker member 340, and the near infrared ray generating member 350 may be attached to a new marker member 340. That is, when the consumable marker member 340 is replaced, the near infrared ray generating member 350 can be reused.

In addition, other effects of the near infrared imaging apparatus 300 according to the third embodiment are the same as those of the near infrared imaging apparatus 100 in the first embodiment.

### MODIFIED EXAMPLES

It should be considered that the embodiments disclosed this time are illustrative in all respects and are not restrictive. The scope of the invention is indicated not by the description of the embodiments but by the scope of claims, and includes meanings equivalent to the scope of claims and all changes (modified examples) within the scope.

For example, in the first to third embodiments, a description has been given of an example in which the near infrared imaging apparatus is configured as the medical imaging apparatus used for angiography and lymphangiography in a surgical operation. However, the invention is not limited thereto. For example, the near infrared imaging apparatus may be configured as an industrial imaging apparatus for product inspection or an imaging apparatus for academic research.

In addition, in the first to third embodiments, a description has been given of an example in which the near infrared imaging apparatus is configured to form the composite image of the near infrared light image and the visible light image. However, the invention is not limited thereto. For example, as in a first modified example illustrated in FIG. 10, a near infrared imaging apparatus 400 may be configured to be able to display only the near infrared light image 92.

Here, as illustrated in FIG. 10, unlike the near infrared imaging apparatus 100 according to the first embodiment, the white light source unit, the visible light sensor, and the image composition unit are not provided in the near infrared imaging apparatus 400 of the first modified example of the first to third embodiments. In this way, only the near infrared light image 92 is displayed on the display unit 9. In this case, when the marker member 40 capable of generating near infrared light is used, the image indicating the position of the marker member 40 is displayed as the near infrared light image 92 on the display unit 9. Thus, even when a composite image of the near infrared light image 92 and the visible light image is not formed, it is possible to inhibit the position at which the mark is actually placed from being shifted from the position at which the mark needs to be placed.

In addition, the first to third embodiments show an example in which the marker light source unit or the marker-side fluorescent portion is disposed around the pen tip of the marker member. However, the invention is not limited thereto. For example, the marker light source unit or the marker-side fluorescent portion may be provided on an opposite side from the pen tip of the marker member.

In addition, the first to third embodiments show an example in which the wavelength of the near infrared light IR1 and the wavelength of the near infrared fluorescence IR3 are configured to be wavelengths in the vicinity of the wavelength of the near infrared fluorescence IR2. However, the invention is not limited thereto. That is, it is sufficient that the wavelength of the near infrared light IR1 and the wavelength of the near infrared fluorescence IR3 are wavelengths that can be detected by the near infrared sensor. The wavelength of the near infrared light IR1 and the wavelength of the near infrared fluorescence IR3 maybe different from the wavelength of the near infrared fluorescence IR2.

In addition, the first embodiment and the third embodiment show an example in which the marker light source unit irradiating the near infrared light IR1 includes the light emitting diode. However, the invention is not limited thereto. That is, the marker light source unit may include a light emitting member other than the light emitting diode. For example, a light bulb may be provided or a laser diode may be provided in the marker light source unit.

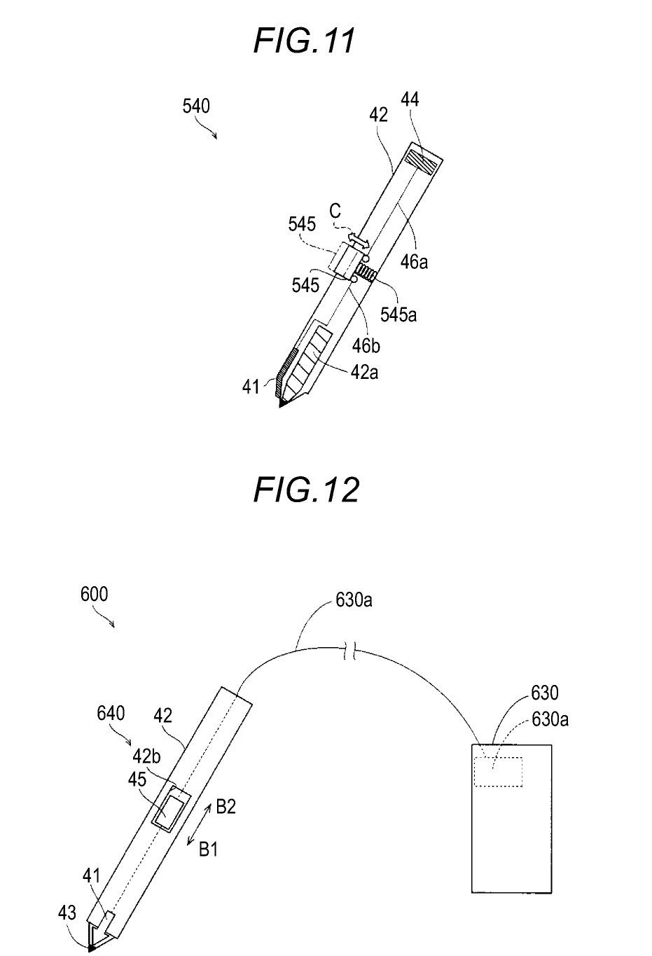

In addition, the first embodiment and the third embodiment show an example of configuring the switch to be slidable along the direction in which the main marker body extends, thereby switching between the state in which power is supplied to the marker light source unit and the state in which power is not supplied to the marker light source unit. However, the invention is not limited thereto. For example, as in a marker member 540 of a second modified example illustrated in FIG. 11, a push button 545 may be provided on the marker member 540.

Here, as illustrated in FIG. 11, in the second modified example of the first embodiment and the third embodiment, the marker member 540 is provided with the push button 545 and an urging member 545a movable in a direction of an arrow C. Further, the marker member 540 is configured such that power is supplied from the battery 44 to the marker light source unit 41 when the push button 545 is pushed to the main marker body 42 side by the user, and the urging member 545a pushes up the push button 545 to the outside of the main marker body 42 and power is not supplied from the battery 44 to the marker light source unit 41 when the push button 545 is not pushed.

In addition, the first embodiment and the third embodiment show an example in which the marker member is provided with the battery for supplying power to the marker light source unit. However, the invention is not limited thereto. For example, as in a near infrared imaging apparatus 600 according to a third modified example illustrated in FIG. 12, power may be supplied from a power supply unit 630a of a main apparatus body 630 to a marker light source unit 41 of a marker member 640 through a cable 630a.

In addition, the second embodiment shows an example in which the marker-side fluorescent agent 241a and the fluorescent agent inside the test object are made of indocyanine green. However, the invention is not limited thereto. That is, the marker-side fluorescent agent 241a and the fluorescent agent inside the test object may be made of a fluorescent agent other than indocyanine green. For example, at least one of the marker-side fluorescent agent 241a and the fluorescent agent inside the test object may be made of 5-ALA.

In addition, the second embodiment shows an example in which the member 241b containing (coated with) the marker-side fluorescent agent 241a is provided in the marker fluorescent portion 240. However, the invention is not limited thereto. For example, a storage container that stores the marker-side fluorescent agent 241a may be provided in the marker fluorescent portion 240, and the storage container may be configured to transmit the near infrared fluorescence IR3 from the inside of the container to the outside of the container.

In addition, the third embodiment shows an example in which the marker-side light source units 352a and 352b are provided in the near infrared light emitting member 350. However, the invention is not limited thereto. For example, a marker-side fluorescent portion having a marker-side fluorescent agent may be provided in the near infrared light emitting member 350.

In addition, the third embodiment shows an example in which the detachable portion 351 is configured to have the shape of the pair of hooks. However, the invention is not limited thereto. For example, an adhesive attachable to and detachable from the main marker body 342 may be provided in the near infrared light emitting member 350.

### REFERENCE SIGNS LIST

1a White light source unit (third light source unit)

1b Excitation light source unit (first light source unit)

5 Visible light sensor (visible light detector)

6 Near infrared sensor (near infrared detector)

7 Image formation unit (imaging unit)

8 Image composition unit (imaging unit)

30, 630 Main apparatus body

40, 240, 340, 540, 640 Marker member

41, 352a, 352b Marker light source unit (second light source unit, near infrared ray generator)

42, 242, 342 Main marker body (holding portion)

43 Pen tip

45, 545 Switch (switching unit)

91 Visible light image

92 Near infrared light image

93 Composite image

100, 200, 300, 400, 600 Near infrared imaging apparatus (medical imaging apparatus, intraoperative support apparatus)

241 Marker-side fluorescent portion (near infrared ray generator)

241a Marker-side fluorescent agent

350 Near infrared ray generating member (near infrared ray generator)

351 Detachable portion

1-12. (canceled)

13. A near infrared imaging apparatus comprising:
a first light source unit that irradiates a test object-side fluorescent agent inside a test object with near infrared excitation light;
a marker member that includes a near infrared ray generator for generating first near infrared light and is used to place a mark on the test object;
a near infrared detector that detects the first near infrared light and detects second near infrared light generated from the test object-side fluorescent agent by the irradiated near infrared excitation light; and
an imaging unit that images the first near infrared light and the second near infrared light detected by the near infrared detector.

14. The near infrared imaging apparatus according to claim 13,
wherein the marker member includes a holding portion corresponding to a portion held by a user and a pen tip disposed on the test object, and
the near infrared ray generator is provided around the pen tip of the marker member.

15. The near infrared imaging apparatus according to claim 13, wherein the near infrared ray generator is formed to have at least one of an arrow shape, a rectangular shape, or a circular shape.

16. The near infrared imaging apparatus according to claim 13, wherein the near infrared ray generator includes a second light source unit that irradiates the near infrared detector with the first near infrared light.

17. The near infrared imaging apparatus according to claim 16, further comprising
a switching unit that switches between a state in which power is supplied to the second light source unit and a state in which the power is not supplied to the second light source unit.

18. The near infrared imaging apparatus according to claim 13, wherein the near infrared ray generator includes a marker-side fluorescent agent that generates the first near infrared light by being irradiated with the near infrared excitation light from the first light source unit.

19. The near infrared imaging apparatus according to claim 13, wherein the near infrared ray generator includes a detachable portion for attachment and detachment to and from the marker member.

20. The near infrared imaging apparatus according to claim 13, wherein a wavelength of the first near infrared light is a wavelength in a vicinity of a wavelength of the second near infrared light.

21. The near infrared imaging apparatus according to claim 13, wherein the near infrared imaging apparatus is used as a medical imaging apparatus.

22. The near infrared imaging apparatus according to claim 13, wherein the near infrared imaging apparatus is used as an intraoperative support apparatus.

23. The near infrared imaging apparatus according to claim 13, further comprising:
a third light source unit that irradiates the test object with visible light; and
a visible light detector that detects the visible light irradiated by the third light source unit and reflected by the test object,
wherein the imaging unit includes an image composition unit that composes a near infrared light image obtained by imaging the first near infrared light and the second near infrared light detected by the near infrared detector and a visible light image obtained by imaging the visible light detected by the visible light detector.

24. A marker member for a near infrared imaging apparatus, the marker member being used for the near infrared imaging apparatus, the near infrared imaging apparatus including a first light source unit that irradiates a test object-side fluorescent agent inside a test object with near infrared excitation light, a near infrared detector that detects second near infrared light generated from the test object-side fluorescent agent by the irradiated near infrared excitation light, and an imaging unit that images the second near infrared light detected by the near infrared detector,
wherein the marker member includes
a near infrared ray generator that generates first near infrared light detectable by the near infrared detector.
